(19) United States
(12) Patent Application Publication
(10) Pub. No.: US 2014/0190888 A1
(43) Pub. Date: Jul. 10, 2014

(54) Methods, Systems and Devices for Separating Tumor Cells

(71) Applicant: VIATAR LLC, Lowell, MA (US)

(72) Inventors: Cornelis Johannes Maria van RIJN, Hengelo (NL); Jacob BAGGERMAN, Wageningen (NL); Ilan REICH, Short Hills, NJ (US)

(73) Assignee: VIATAR LLC, Lowell, MA (US)

(21) Appl. No.: 14/163,344

(22) Filed: Jan. 24, 2014

Related U.S. Application Data
(63) Continuation of application No. 13/077,427, filed on Mar. 31, 2011, now abandoned.

(30) Foreign Application Priority Data
Mar. 31, 2010 (NL) 1037837
Nov. 4, 2010 (NL) 1038359

(51) Int. Cl. A61M 1/34

(52) U.S. Cl. CPC A61M 1/34
USPC 210/651

(57) ABSTRACT

Embodiments of the present disclosure are directed to the separation/capture of specific cells and/or contaminants, as well as the determination, monitoring, and treatment of cancer. Moreover, some embodiments are directed to methods, systems and devices for removing cancer, stem and/or tumor cells in vivo or in vitro from a bodily fluid to prevent or impede the proliferation of a cancer. Some embodiments provide a blood-compatible filter comprising, for example, a membrane provided with a number of openings (preferably precise) which yield minimal detrimental effect both quantitatively and qualitatively on cells present in the bodily fluid during the separation process. For example, in some embodiments, a majority percentage of circulating tumor cells are captured by a filter while a majority percentage of leukocytes, for example, are allowed to pass, where the passed leukocytes retain their vitality.

### CROSS-REFERENCE TO RELATED APPLICATIONS

The present application is a continuation of U.S. application Ser. No. 13/077,427, filed Mar. 31, 2011, which claims the benefit of and priority to Netherlands' patent application nos. NL1037837, entitled, “Device and Method for Separation of Circulating Tumor Cells,” filed Mar. 31, 2010, and NL1038359, entitled, “Device and Method for Separation of Circulating Tumor Cells,” filed Nov. 4, 2010, all of which are hereby incorporated by reference herein in their entireties.

### FIELD

Embodiments of the present disclosure are directed to methods, systems and devices for at least one of, and in some embodiments both of, separating and counting circulating tumor cells (CTCs) from blood.

### BACKGROUND

Metastasis of a primary cancer is believed to begin when cancer cells (circulating tumor cells, or CTCs) migrate from the primary cancer into the peripheral blood and/or lymph circulation. Removal of these CTCs is therefore important. Although a CTC may eventually be trapped by a blood capillary or a lymph node it is also known that CTCs are able to travel a number of times through the circulatory system.

It is also important, aside from any diagnostic or therapeutic reasons to remove CTCs, to capture CTCs for analysis, including any experimentation for drug discovery/development and the like. Thus, capturing the CTCs from a bodily fluid for later use is also important.

The separation and counting of circulating tumor cells from blood can be used to clinically assess a metastatic cancer and also to monitor therapeutic effects of various treatment modalities. Current techniques for separating and counting CTCs from blood are based on either magnetic bead separation, density-gradient centrifugation, and filtering methods, or combinations thereof.

While the use of bio-functionalized surfaces (e.g., selectin CD62) has been shown to catch or adhere CTCs, such surfaces have the disadvantage that only a specific fraction of the cancer cells can be obtained, and only for a specific time. Moreover, proteins and other functional cells may adhere to bio-functionalized surfaces which may trigger immune reactions.

### SUMMARY

Some of the embodiments of the present disclosure provide methods, systems and/or devices for any and all of: separating CTCs from a fluid, separating contaminants from a fluid (e.g., any cell type, including bacteria and virus cells), separating CTCs/contaminants from a bodily fluid, separating CTCs/contaminants from blood, and separating CTCs/contaminants from at least one of untreated and unprocessed blood. In any of the foregoing, some embodiments of the disclosure present methods, systems and devices not only to separate CTCs/contaminants, but while doing so, also preserving the vitality of at least one of components, cells (e.g., red blood cells, while blood cells/leukocytes, platelets, bacteria, viruses). Some embodiments present methods, systems and/or devices, for at least one of assessing, monitoring and treating one or more cancers. Moreover, in any and all embodiments, processes (and the systems and/or devices for carrying out such processes) may accomplish any and all of the noted functionality either or both of in vivo or in vitro. Such ability, according to some embodiments, may aid in at least one of impeding, preventing and treating disease, e.g., cancer (and/or the proliferation thereof).

Accordingly, it is an object of at least some of the embodiments of the present disclosure to trap and/or capture CTCs in a bodily fluid, e.g., a blood sample. In some embodiments, it is an object to capture CTCs which are traveling through the circulatory system, such that, proliferation of the cancer may be prevented or at least impeded.

Capture may be defined as separation of target particles (e.g. cells) from fluid by use of a filter that separates the particles by at least one of retention and binding of the target particles to a surface of a membrane having a predetermined thickness and openings of at least one of a predetermined size, shape and arrangement on the membrane. According to some embodiments, capture may include retention and binding of the target particles to a coating of the surface of the membrane, which may include, for example, affinity bodies (e.g., antibodies).

It is an object of at least some of the embodiments of the present disclosure to remove cancer cells via at least one of in vivo and in vitro from a bodily fluid (sample, or direct from patient) in order to prevent, impede the proliferation of the cancer (and/or treat the cancer) with minimal detrimental effect on the presence of any other cells, both quantitatively and qualitatively, in the bodily fluid. In some embodiments, the filtered bodily fluid may be directed back to the patient from which the bodily fluid came from, and/or stored for any of: experimentation, use in the patient or another patient, analysis, and the like.

It is another object of at least some of the embodiments of the disclosure to provide methods, systems and/or devices to at least one of clinically assess and monitor a therapeutic effect with respect to a targeted cancer.

It is another object of at least some of the embodiments of the disclosure to provide a real time, non-invasive, extracorporeal liquid biopsy, with substantially no material loss of patient's blood (and in some embodiments, no material loss of a patient's blood) to trap and/or capture a statistically significant quantity of cells (e.g., 10⁵), which can then be used for drug trial validation, therapeutic decisions, genetic research, and/or other related diagnostic and/or therapeutic methods. For example: Phosphatidylinositol 3-kinases (PI 3-kinases or PI3Ks) are a family of enzymes involved in cellular functions such as cell growth, proliferation, differentiation, motility, survival and intracellular trafficking, which in turn are involved in cancer cells. Thus, a liquid CTC biopsy can be used to determine if a mutation in one or more of these enzymes (for example) has occurred in the CTC's. Accordingly, such a determination can be used as a factor to determine an adequate therapy for the patient.

It is a particular feature, according to at least some embodiments of the present disclosure, that the bodily fluid which contains the CTCs need not be pre-treated for filtering, e.g., embodiments of the present disclosure need not enrich, dilute, fixate (e.g. fixating agents as formaldehyde) and the like, to capture or otherwise separate CTCs from a bodily fluid. Known prior art systems for filtering CTCs all require some form of enrichment or cell fixation, i.e., dilution of a patient's blood sample (for example). Such distinguishing features are specifically important in an extracorporeal system since it is impractical to continuously dilute or fixate a patient's blood (for example) to a degree necessary by known prior art systems (e.g., 10:1). As one of ordinary skill in the art will appreciate, such a degree of dilution inherently limits the utility of such systems relative to the amount of blood which can be drawn from a patient at a time (maximum 20 ml). Consequently, such systems can only capture a relatively small number of CTCs compared to embodiments of the present disclosure. See, e.g., “3D microfilter device for viable circulating tumor cell (CTC) enrichment from blood,” Zheng et al., Springer Science+Business Media, LLC, 27 Oct. 2010); and “Isolation of circulating tumor cells using a microvortex-generating herringbone-chip,” Stott et al., PNAS, 26 Oct. 2010; both disclosures of which are herein incorporated by reference in their entireties.

Throughout the present disclosure and as well as recited in the claims, the acronym CTCs (circulating tumor cells), may include any one of the following cells types and/or classifications: cancer cells, tumor cells (malignant or benign), and stem cells. In some embodiments, CTCs may also include bacteria and viruses, contaminants, and/or any targeted particle that is desired to be captured from a bodily fluid, for at least one of storage, analysis, experimentation, diagnosis, therapy and treatment. Accordingly, cancer cells include any tumor, malignant and/or diseased cell.

Moreover, the phrase “bodily fluid”, in addition to covering any bodily fluid of the body, e.g., blood, may also, in some embodiments, mean any sample fluid which contains cancer cells for capture.

In some embodiments, an important feature is the passing of a majority percentage, and preferably all, or substantially all, of leukocytes (which may also be referred to as “white blood cells”, the phrase used interchangeably with leukocytes throughout the present disclosure) contained in the bodily fluid (e.g., blood), and retaining the vitality of all or substantially all of the passed leukocytes, while capturing (or otherwise filtering, retaining, separating) all or substantially all of the CTCs in the bodily fluid. In some therapeutic embodiments, such functionality enables the preservation and/or enhancement of the immune system of a patient.

Moreover, in some embodiments, captured CTCs can be fused with dendritic cells (e.g., from a cell line) to create hybrid cells which may then be used to activate the patient's immune system (i.e., the fused CTC/dentritic cells are placed back into the patient). When the hybrid cells are given to the patient, the cells are expected to express a spectrum of patient's tumor specific antigens. In the case where the immune system of the patient has enough healthy white blood cells, there is a greater chance that the immune system of the patient will produce an adequate response to kill the cancer cells. To that end, it is expected that at least 100,000 hybrid cells are required for this to occur. Accordingly, the device according to some embodiments of the disclosure can harvest a relatively large number of CTCs (e.g., greater than 100,000) from blood. Moreover, in some embodiments, the CTCs are captured by at least one of retention, and binding to tumor specific antigens attached to the surface of the membrane.

In some embodiments, a method for fusion to produce hybrid cells from the captured CTCs is provided. For example, a membrane with CTCs (e.g., approximately 100,000 or greater) is placed on the bottom of a cell fusion chamber with the membrane surface (having the CTCs) facing up. Preferably, an equal number of dendritic cells are fed into the chamber. The dendritic cells slowly deposit (by at least one of gravity and filtration via the openings in the membrane) on top of the CTCs. Next, an adequate RF pulse train (as known in the art) is applied to fuse the dendritic cells with the CTCs, which results in the formation of hybrid cells. For example, a membrane with the cells is subject to an alternating field, e.g., about 250-300 V/cm at 1 MHz (for example) to stabilize the cell suspension. Next, a fusion pulse of sufficient amplitude (for example, about 1500 V/cm) and duration (for example, about 30-50 μs), is applied. After the fusion pulse, an alternating field of the same frequency is applied again to maintain contact between the cells during the mixing of cytoplasms and reconstruction of the membrane around the bi-nucleated hybrid cells.

In some embodiments, especially with respect to diagnostic embodiments, the feature of passing a majority of the leukocytes (and as previously noted, preferably substantially all leukocytes, and most preferably, all leukocytes) is an important feature in that DNA analysis of captured CTCs is critical in assessing the cancer of the patient and formulating a treatment. Accordingly, having leukocytes on the filter/membrane is less desirable.

In some embodiments, a method for separating CTCs from a bodily fluid of a patient while preserving leukocytes contained in the bodily fluid is provided. The method includes providing a filter having a flow capacity, flowing a bodily fluid including at least a plurality of CTCs and a plurality of leukocytes through the filter, capturing, by the filter, a majority percentage of the CTCs contained in the bodily fluid, and passing a majority percentage of the leukocytes through the filter, wherein the vitality of substantially all of the passed leukocytes is preserved.

In such embodiments, for example, the majority percentage of at least one of the captured CTCs and the passed leukocytes is selected from the group consisting of: greater than about 75%, greater than about 80%, greater than about 85%, greater than about 90%, greater than about 95%, greater than about 99%, and greater than about 99.9%.

Furthermore, in such embodiments, the filter is initially optimized to capture a first type of CTC, and a majority percentage of the first type of CTCs present in the bodily fluid are captured. Such optimization may comprise filtering a first sample of a predetermined quantity of the bodily fluid at a predetermined filtering pressure using a first filter having a predetermined filtering area and filter openings of a predetermined quantity per unit of filtering area and predetermined width, capturing CTCs contained in the predetermined quantity of the bodily fluid by the first filter, determining a quantity of the CTCs captured, the quantity associated with a capture percentage, and repeating filtering of CTCs from a second sample of a predetermined quantity of bodily fluid upon the capture percentage being less than a predetermined capture percentage, wherein for subsequent iterations of filtering, at least one of filtering pressure, filtering area, filter opening quantity per unit of filtering area, and filter opening width is modified from a previous iteration of filtering. In some embodiments, fixated CTCs show a higher capture efficiency for a given filter type.

Likewise, in some embodiments, captured un-fixated CTCs can be inactivated by pushing them with a sufficiently high pressure (e.g. 50-500 mbar) through the membrane with openings of a relatively narrow width (e.g., about 3-6 micrometers). Pulses may be applied, for example, for about every second for about a 15 minute interval to press CTCs through the membrane openings, and therefore could be used to clear unwanted CTCs captured from blood from the membrane during a therapy session (for example). In some embodiments, it is preferable to keep pulse duration relatively short (as possible), to minimize any possible detrimental effects on other blood cells. Accordingly, some embodiments of an extracorporeal system for capturing CTCs from the blood of a patient using such a feature is also presented by this disclosure.

In some embodiments, CTCs captured on a membrane surface can be inactivated/killed by using an electrically conductive boron or phosphorous doped diamond like carbon membrane (DLC). These conductive membranes can be subjected to relatively high electrical voltage pulses without degradation of the material to make strong radical molecules that will attack all organic species present on the conductive membrane surface. At mild voltage pulses, CTC present at the membrane surface will be deactivated (e.g. during a therapy session), while driving at high voltage even total cleaning of the membrane can be achieved (e.g. for sterilization).

According to some embodiments, the filter comprises a membrane including a thickness and including a plurality of openings arranged on the membrane and passing through the membrane. In some embodiments, the thickness of the membrane and the width of the openings are preferably configured to capture the majority percentage of the CTCs and/or other contaminants and pass and preserve the vitality of the majority percentage of leukocytes and/or other “good” components in the bodily fluid.

In some embodiments, a method for preserving and/or enhancing the immune system of a cancer patient is provided and includes directing a flow of a predetermined amount of blood of a cancer patient to a filter, capturing, by the filter, a majority percentage of the CTCs contained in the blood, passing a majority percentage of the leukocytes through the filter, wherein the vitality of substantially all of the passed leukocytes is preserved, and directing the filtered blood contained the passed leukocytes back to the patient. Similar to previous embodiments, the majority percentage of at least one of the captured CTCs and the passed leukocytes is selected from the group consisting of: greater than about 75%, greater than about 80%, greater than about 85%, greater than about 90%, greater than about 95%, greater than about 99%, and greater than about 99.9%.

In some embodiments, a system for capturing CTCs from a bodily fluid also at least containing leukocytes is provided and includes a pump, a filter having an inlet and an outlet, a first conduit to establish fluid communication between a source of bodily fluid and a the filter, and a second conduit to establish fluid communication between the filter and the pump. The filter is configured to capture a majority percentage of the CTCs contained in the bodily fluid and pass a majority percentage of the leukocytes through the filter, wherein the vitality of substantially all of the passed leukocytes is preserved.

In such embodiments, any and all of the following may be additionally included: a first pressure sensor to determine a pressure in the first conduit, and a second pressure sensor to determine the a pressure in the second conduit. The pump is selected from the group consisting of: a peristaltic pump, a gear pump, a progressive cavity pump, a roots-type, a venturi pump, a piston/reciprocating pumps, a compressed gas/air pumps, and a combination of any of the forgoing.

In any system embodiment presented by the subject disclosure (and even device components), may also be included a controller for controlling operation and/or monitoring at least one of flow and pressure of the device/system.

In some embodiments, an extracorporeal system for capturing CTCs from the blood of a patient is provided and includes a controller optionally including a pump loop response timer, a pump, a filter, a first conduit to establish fluid communication between the supply of blood from the patient and the pump, a second conduit to establish fluid communication between the pump and the filter, and a third conduit for providing fluid communication out of the filter. The filter is configured to capture a majority percentage of the CTCs contained in the blood and pass a majority percentage of the leukocytes through the filter, wherein the vitality of substantially all of the passed leukocytes is preserved.

In such embodiments, the third conduit establishes fluid communication between the filter and the patient or a container, and may also include a valve, where the third conduit provides fluid communication between the filter and the valve. In still further embodiments, a fourth conduit may be provided for such a system for establishing fluid communication between the valve and the patient, where filtered blood is delivered back to the patient.

Such embodiments may further include at least one pressure sensor for monitoring pressure of fluid communication into the filter assembly, or at least two pressure sensors, one pressure sensor to monitor pressure of fluid communication between the patient and the pump, and a second pressure sensor to monitor pressure of fluid communication between the pump and the filter. Still further, such systems may additional comprise third and fourth pressure sensors, the third pressure sensor to monitor pressure between the filter and the valve, and the fourth pressure sensor for monitoring pressure between the valve and the patient.

Bubble sensors may also be additionally provided to sense bubbles in the bodily fluid in, for example, the third conduit.

Any and all embodiments of the present disclosure may further include one or more counting devices for counting or otherwise characterizing the captured contaminants, CTCs, etc. Such counting devices/systems that may include, for example: CASY cell counters, and Coulter counters (see also, U.S. Pat. Nos. 7,738,094, 7,136,152, 6,974,692, 6,350,619, 5,962,238, 5,556,764, 4,296,373, and 3,977,995, for example; each of the forgoing references herein incorporated by reference in their entirety).

In some embodiments, a method for separating cancer cells from a bodily fluid utilizing a separation system is provided, where the separation system includes a controller, a pump for providing a first directed flow-rate, a filter, a first conduit to establish fluid communication between a source of bodily fluid and the filter, a second conduit to establish fluid communication between the filter assembly and the pump, a first pressure sensor for monitoring a first pressure P1 of fluid communication in the first conduit, and a second pressure sensor for monitoring a pressure P2 of fluid communication in the second conduit, and a controller for controlling operation of at least the pump. The method may include measuring pressure P1 and P2 at predetermined time intervals; where for each time interval the method further includes determining a differential pressure value between P1 and P2, and comparing the differential pressure value to a predetermined target pressure range. The target pressure range comprises a target pressure value±a pressure hysteresis value. In such embodiments, upon the input differential pressure value being within the target pressure range, the first directed flow-rate of the pump is unchained and the process returns to measurement of pressures P1 and P2 for a subsequent time interval, and upon the input differential pressure value being outside the target pressure range, a new pump flow-rate is determined and the first directed flow-rate of the pump is changed to the new pump flow-rate, and the process returns to measurement of the pressures P1 and P2 for a subsequent time interval.

In such embodiments, the system further comprises a pump loop response timer, where the pump loop response timer operates in countdown fashion, and where calculation of a new pump flow-rate comprises: upon detection of the input differential pressure value being greater than the sum of the target pressure value and a pressure hysteresis value, the new pump flow rate comprises a reduction of the first directed pump flow-rate by the flow rate step size. Moreover, upon detection of the input differential pressure value being less than the difference between of the target pressure value and a pressure hysteresis value, the new pump flow rate comprises an increase of the first directed pump flow-rate by the flow rate step size. In such embodiments, the flow rate step size is selected to eliminate overshoot.

In some embodiments, a method for diagnosing cancer and/or a type of cancer is provided and includes providing a filter having a flow capacity, flowing a bodily fluid including at least a plurality of CTCs and a plurality of leukocytes through the filter, capturing, by the filter, a majority percentage of the CTCs contained in the bodily fluid, passing a majority percentage of the leukocytes through the filter, wherein the vitality of substantially all of the passed leukocytes is preserved, performing analysis of captured CTCs, and determining cancer and/or a type of cancer of the CTCs.

In some embodiments, a method of cancer treatment is provided and includes providing a filter having a flow capacity, flowing a bodily fluid including at least a plurality of CTCs and a plurality of leukocytes through the filter, capturing, by the filter, a majority percentage of the CTCs contained in the bodily fluid, passing a majority percentage of the leukocytes through the filter, wherein the vitality of substantially all of the passed leukocytes is preserved, performing analysis of captured CTCs, determining cancer and/or a type of cancer of the CTCs, and determining a treatment for the determined cancer.

In some embodiments, a method for preserving and/or enhancing an immune system of a patient is provided and includes providing a filter having a flow capacity, flowing a bodily fluid including at least a plurality of contaminants and a plurality of leukocytes through the filter, capturing, by the filter, a majority percentage of the contaminants contained in the bodily fluid, passing a majority percentage of the leukocytes through the filter, wherein the vitality of substantially all of the passed leukocytes is preserved, and directing the filtered bodily fluid containing the passed leukocytes back into the patient.

In some embodiments, a method for separating contaminants from a bodily fluid of a patient while preserving leukocytes contained in the bodily fluid is provided, and includes providing a filter having a flow capacity, flowing a bodily fluid including at least a plurality of contaminants and a plurality of leukocytes through the filter, capturing, by the filter, a majority percentage of the contaminants contained in the bodily fluid, and passing a majority percentage of the leukocytes through the filter, where the vitality of substantially all of the passed leukocytes is preserved.

In some embodiments, a method for CTC/dendritic cell fusion is provided and includes providing a membrane with a quantity of CTCs, placing the membrane with the CTCs at the bottom of a cell fusion chamber with the membrane surface having the CTCs facing up, feeding at least a corresponding amount of dendritic cells into the chamber, wherein the dendritic cells deposit on the CTCs, and applying an RF pulse sequence (e.g., see above), where the dendritic cells fuse with the CTCs to form hybrid cells.

In some embodiments, a method for coating a CTC filter comprising a membrane is provided and includes providing a membrane having a first surface of silicon nitride, the silicon nitride containing at least one of Si—H and NH₂ functionalities, where a layer of silicon oxide is present on the first surface. The method may also include removing the silicon oxide layer on a first surface of the membrane, and reacting the silicon nitride surface with a compound containing at least one of a terminal alkene or alkyne moiety for direct covalent attachment to the surface via Si—C bonds.

Accordingly, many other embodiments are possible, including a multitude of therapeutic and diagnostic embodiments. For examples, some embodiments of the present disclosure include the capture of CTCs from a bodily fluid, analyzing the captured CTCs genetically, determining the type of cancer and/or determining a treatment. Determining a treatment may be any treatment available for the determined cancer. Thus, embodiments of the present disclosure include methods for determining cancer (and/or type of cancer), methods for determining treatment of a cancer, and methods for treating cancer, using, for example, filtering/separation features of some of the embodiments of the present disclosure.

The openings of membranes according to some embodiments may be between about 3 μm and about 5 μm, and in some embodiments may include a width between about 5 μm and about 8 μm.

One or more of the above-noted embodiments with respect to any and all of methods, systems, and devices disclosed herein, as well as any other embodiment which is supported by the present disclosure, may include one or more of the following features:
optimization of the filter to capture a first type of CTC, and a majority percentage of the first type of CTCs present in the bodily fluid are captured;
such optimization (as indicated above) may include one or more (and preferably several or all) of: filtering a first sample of a predetermined quantity of the bodily fluid at a predetermined filtering pressure using a first filter having a predetermined filtering area and filter openings of a predetermined quantity per unit of filtering area and predetermined width, capturing CTCs contained in the predetermined quantity of the bodily fluid by the first filter, determining a quantity of the CTCs captured, the quantity associated with a capture percentage, and repeating filtering of CTCs from a second sample of a predetermined quantity of bodily fluid upon the capture percentage being less than a predetermined capture percentage. For subsequent iterations of filtering, at least one of filtering pressure, filtering area, filter opening quantity per unit of filtering area, and filter opening width is modified from a previous iteration of filtering.
the thickness of the membrane and the width of the openings are configured to capture the majority percentage of the CTCs and pass and preserve the vitality of the majority percentage of leukocytes in the bodily fluid;
the bodily fluid is not treated prior to being flowed past a filter;
the bodily fluid is not treated with fixating agents prior to being flowed past the filter;
methods where the majority percentage of at least one of the captured CTCs and passed leukocytes is selected from the group consisting of: greater than about 75%, greater than about 80%, greater than about 85%, greater than about 90%, greater than about 95%, greater than about 99%, and greater than about 99.9%;
pressure sensor(s) to determine the a pressure along any and all fluid conduits;
pumps selected from the group consisting of: a peristaltic pump, a gear pump, a progressive cavity pump, a roots-type, a venturi pump, a piston/reciprocating pumps, a compressed gas/air pumps, and a combination of any of the forgoing;
one or more controller(s), processors, monitors, sensors, memory, communications, and circuitry for controlling operation, reporting, communications, and/or monitoring of any method, system and/or device disclosed herein, either via analog, digital or a combination thereof;
one or more fluid conduits for establishing fluid communications between any disclosed element (e.g., filter, pump, sensor, container, patient, valves, and the like);
one or more valves;
pressure and/or bubble sensors, provided anywhere in a system (e.g., pump, filter, conduit, valve);
one or more counting devices provided anywhere in a system/device according to some embodiments, for counting or otherwise characterizing at least one of captured CTCs, contaminants and passed cells (e.g., leukocytes);
filtering membranes which include functionalized antibodies and/or receptor molecules configured to adhere to at least a part of one or more CTCs, where such receptor molecules may be configured on a zwitterionic coating to avoid non-selective adsorption of other species;
timers, for example, a pump loop response timer, where such a time operates in countdown fashion;
methods, systems and devices for calculating a new pump flow-rate, which may include one or more of (and preferably several or all of): upon detection of an input differential pressure value being greater than the sum of the target pressure value and a pressure hysteresis value, the new pump flow rate comprises a reduction of the first directed pump flow-rate by a flow rate step size, and upon detection of the input differential pressure value being less than the difference between of the target pressure value and a pressure hysteresis value, the new pump flow rate comprises an increase of the first directed pump flow-rate by the flow rate step size; and
a flow rate step size (see above), may be selected to eliminate overshoot.

These and other embodiments, objects and advantage of the methods, systems and devices disclosed in the present application will become even more evident by reference to the following drawings and detailed description which follows.

### BRIEF DESCRIPTION OF DRAWINGS

FIG. 1 illustrates a cross-sectional view of the membrane for capturing cancer cells according to some embodiments of the present disclosure.

FIGS. 2A and 2B illustrates a membrane viewed at 20× magnification for the presence of the cancer cells after separation thereof from a fluid flow, according to some embodiments of the subject disclosure; FIG. 2A illustrating an embodiment of the membrane without a zwitterionic coating and FIG. 2B illustrating an embodiment of the membrane with a zwitterionic coating.

FIG. 3A illustrates a schematic diagram of a system for separating CTCs (and/or other cells, contaminants and the like) from a limited fluid sample, according to some embodiments of the subject disclosure.

FIG. 3B illustrates a perspective view of an exemplary system of the schematic shown in FIG. 3A for separating CTCs from a limited fluid sample, according to some embodiments of the subject disclosure.

FIG. 4A illustrates a schematic diagram of an extracorporeal system for separating CTCs (and/or other cells, contaminants and the like) from a large fluid sample (e.g., a significant amount of blood directly/indirectly from a patient), according to some embodiments of the subject disclosure.

FIG. 4B illustrates a perspective view of an extracorporeal system of the schematic shown in FIG. 4A for separating CTCs from a large fluid sample, according to some embodiments of the subject disclosure.

FIG. 5 illustrates an exemplary process flow for controlling a flow of fluid sample (e.g., bodily fluid) through the exemplary system provided in FIGS. 3A-B, according to some embodiments of the subject disclosure.

FIGS. 6A and 6B show a table illustrating the distribution of Epithelial Cell Adhesion Molecule (Ep-CAM) found in normal human adult tissues (see, e.g., Balzar, M., et al., “The Biology of the 17-1A antigen (Ep-CAM),” J. Mol. Med., 77:699-712 (1999).

FIG. 7 is a table illustrating Ep-CAM expression in human malignant neoplasias (see, e.g., Balzar, M., et al., “The Biology of the 17-1A antigen (Ep-CAM),” J. Mol. Med., 77:699-712 (1999).

FIGS. 8A and 8B are enlarged photographs (of different magnification) of a filter membrane having openings arranged along the surface thereof, according to some embodiments of the present disclosure.

### DETAILED DESCRIPTION

At least some embodiments of the present disclosure provide methods, systems and devices for separating (which may also be referred to as capturing or filtering, separating, capturing and filtering used interchangeably throughout the present disclosure) a majority percentage of CTCs contained in a bodily fluid (e.g., blood), where such embodiments include a blood-compatible filter. Such a filter may comprise a membrane or similar structure provided with a number of openings (e.g., a micro-sieve/filter; openings may also be referred to as pores or pore in the singular), and in some embodiments, the openings are precise. That is, the tolerance of the openings, according to some embodiments, are within: less than about 0.5 μm, less than about 0.25 μm, less than about 0.1 μm, less than about 0.05, less than about 0.025, and less than about 0.01 μm. It is worth noting that membrane, in some embodiments, can be any generally thin, flat, plate-like structure having a thickness, including, for example, hollow fiber, tracked etch membranes, micromachined membranes, PDMS membranes, etc.; such materials may be either a single layer or sophisticated/complex structures (e.g., three-dimensional).

According to some embodiments, a majority percentage includes, but is not limited to greater than about: 50%, 55%, 60%, 65%, 70%, 75%, 80%, 85%, 90%, 95%, 99%, 99.9%, and 99.99%, and values in between such percentages (for at least one of: capture of CTCs and/or other predetermined specific cells and/or contaminants, and passage of leukocytes and/or other vital components of the filtered medium (i.e., bodily fluid).

In some embodiments, the openings provided over the membrane have minimal effect, and in some embodiments, minimal detrimental effect, both quantitatively and qualitatively, on normal cells and/or components present in the bodily fluid during the separation/filtration process (for embodiments of the present disclosure, separation and filtration are used synonymously), and in particular, on leukocytes present in the bodily fluid. Moreover, at least in some of the disclosed embodiments, at least one of the following is a result of the filtering of bodily fluid: substantially no hemolysis, substantially no blood platelet damage and/or activation, and substantially no leukocyte damage, activation and/or retention occurs. In some embodiments, at least one of the following is a result of filtering of bodily fluid: no hemolysis, no blood platelet damage and/or activation and no leukocyte damage, activation and/or retention occurs, as well as complement system activation, coagulation system activation, thrombosis, etc. (e.g., ISO requirements for testing extracorporeal blood systems).

Qualitatively and quantitatively means, in some embodiments for example, that the passage of red blood cells through the membrane with a hemolysis less than about 1%, less than about 0.8%, less than about 0.5%, or less than about 0.1% (depending upon the embodiment). Qualitatively and quantitatively means, according to some embodiments, for example, that the membrane is capable of retaining CTCs in a majority percentage, including, for example: 75%, 80%, 85%, 90%, 95%, 99%, 99.9% and 99.99%; and allowing the passage of a majority percentage of blood platelets more than about 90%, and in other embodiments: preferably more than: about 95%, about 99%, about 99.9%, and about 99.99% of the blood platelets without any noticeable platelet activation and values in between such percentages. Moreover, in some embodiments, qualitatively and quantitatively means, for example, that the membrane is capable of retaining CTCs and allowing the passage of more than about 95% of white blood cells with minimal to substantially no damage to the white blood cells and/or with minimal to substantially no white blood cell activation. Moreover, according to some embodiments, such a percentage may be: about 99%, about 99.9%, and about 99.99% (depending upon the embodiment). For such embodiments, qualitatively may also connote that the vitality of a majority percentage of the leukocytes which are passed by the filter is preserved (e.g., healthy, functioning). According to some embodiments, a majority percentage of passed leukocytes includes, but is not limited to greater than about: 95%, 99%, 99.9%, 99.99%, 99,9999 and 99.999999% and values in between such percentages (generally, the maximum number of leukocytes retained is less than the number of openings in the membrane as the actual number of leukocytes passed is many orders or magnitude greater). Accordingly, the vitality of cells (e.g., passed leukocytes) may be defined as healthy, adequate and/or good cells which are able to contribute to their intended functionality within the body of a human or animal, for example.

TABLE 1
Coating Methods (“+” connotes advantageous, and “−” connotes
disadvantageous)

| | | | Dip coating + | CVD + |
|---|---|---|---|---|
| Requirement | SAM | ATRP | curing | modifications |
| Conformal | + | + | − | + |
| Controlled | − | +/− | − | + |
| thickness | | | | |
| Surface | − | − | + | + |
| independent | | | | |
| Solvent free | − | − | − | + |
| Small post | + | + | − | + |
| treatment | | | | |
| roughness | | | | |
| Scalability | − | − | + | + |

Several characteristics of some of the method embodiments available for applying organics coatings on silicon nitride are summarized in Table 1. Ideally, it is preferable that the technique yield a conformal coating with a controlled thickness and low roughness through a surface independent and scalable process. For example, a self-assembled monolayer(s) (SAM) can be used for functionalizing a surface with a conformal organic layer. Such a method generally requires specific reactivity of the organic compound(s) forming the monolayer to match with the intended surface. For example, silicon oxy nitride can be functionalized with organosilanes for monolayer deposition or hydrogen terminated silicon nitride with alkenes or alkynes. Drawbacks of such a process include scalability and thickness control. Thickness control, however, can be improved by using atom transfer radical polymerization (ATRP) for growing a polymer layer, although this still requires a specific reactive group on the surface, i.e., an ATRP polymerization initiator and a solvent based process.

An alternative process for applying coatings to membranes according to some embodiments, is dip-coating, where the surface is dipped in a solution with monomeric or polymeric materials. After removal, a thin film of the solution is left on the surface and dried, resulting in a thin layer of organic material which is subsequently cured. This process is substrate independent and scalable, though offers less control on conformal coating and thickness of the final film. Chemical vapor deposition (CVD) may be utilized for applying a coating to membranes according to some embodiments. CVD is a gas-phase process for applying films on substrates and often used for inorganic materials. More recently methods were developed for CVD of organic films, such as plasma-enhanced CVD, pulsed plasma CVD, hotwire CVD and initiated CVD. These methods allow the deposition of organic polymeric films in a conformal way with a controlled thickness. The advantage of these processes is that they are surface independent, solvent free and scalable.

In some embodiments, methods, systems and/or devices are provided which include a membrane with openings, and which also includes a hemo-compatible coating on the membrane surface with a thickness, preferably of less than about 500 nanometers. Such a hemo-compatible coating according to some embodiments, for example, includes a minimal interaction between the coating material and blood, and preferably without inducing uncontrolled activation of cellular or plasma protein cascades (e.g., by protein adsorption) thus preventing blood coagulation and platelet aggregation. The prevention of the formation of blood clots and protein aggregates is advantageous since the foregoing can block filtration and adversely affects device performance.

In some embodiments, the coating can be an inorganic material, such as titanium, titanium nitride, titanium dioxide, and/or organic materials. The organic materials can be hydrophobic in nature such as polysiloxanes and PTFE (polytetrafluoroethylene) or hydrophilic such as, pHEMA (Poly-2-hydroxyethylmethacrylate) and zwitterionic polymeric materials (polymers containing oppositely charged groups). Also a copolymer containing both hydrophilic and hydrophobic monomers resulting in a polymer film with nanometer sized domains with alternating hydrophilic and hydrophobic domains can be an effective blood contacting material. In some embodiments, it is preferable that the coatings are durable and reusable, as it has been found that well known PEO (polyethyleneoxide) or PEG (polyethyleneglycol) coatings are relatively unstable and such molecules have been found to decompose by further oxidation of the carbon chain within a few days. Accordingly, in some embodiments, PEO and PEG coatings should be avoided.

The coating according to some embodiments comprises zwitterionic groups including molecules such as phosphorylcholine, sulfobetaine, carboxybetaine, or amine-N-oxide subgroups. Membranes according to some embodiments modified with zwitterionic polymers with phosphorylcholine, sulfobetaine or carboxybetaine groups have shown excellent hemocompatibility with respect to hemolysis and blood platelet activation and also prevent clogging of the membrane openings. Because of the close resemblance of the zwitterionic groups with well-known (stabilizing) osmolytes, polymers derived from trimethyl amine-N-oxide, i.e., containing N-oxide groups, for example, will be also effective as hemocompatible coatings.

In some embodiments, in view of endurable application, hemocompatible molecules are covalently attached to the membrane surface. A covalent bond is a form of chemical bonding that is characterized by the sharing of pairs of electrons between atoms. For example, covalent attachment on the native oxide of the silicon nitride is possible using silane or siloxane chemistry. However, these bonds are prone to hydrolysis and therefore attachment via a direct silicon-carbon or nitrogen-carbon bond is preferred and/or optimal with respect to an endurable surface coating on membranes according to some embodiments of the present disclosure. This can be achieved, for example, by removing the silicon oxide layer on top of the membrane and reacting the bare silicon nitride surface, containing Si—H and NH₂ functionalities, with a compound containing a terminal alkene or alkyne moiety for direct covalent attachment to the surface via Si—C bonds. The reaction can be done using at least one of thermal or photochemical activation of the surface and contacting the surface with the reactant from at least one of the gas phase or the liquid phase, i.e., neat liquid compound and/or dissolved solvent. Similarly, the NH₂ groups of the bare silicon nitride can be used for covalent attachment of organic groups using for example alkyl halides, aldehydes, anhydrides and acid halide groups can be used to functionalize the surface. The compound can contain another functional group(s), such as alkene, carboxylic acid, ester, amide, N-hydroxy succinimide or epoxide, rendering the surface suitable for further functionalization of the surfaces.

In a similar fashion as silicon nitride, according to some embodiments, hydrogen terminated diamond-like carbon membranes can be coated with a compound containing a terminal alkene or alkyne moiety for direct covalent attachment to the surface via C—C bonds. Diamond like carbon can also functionalized by treating the surface, for example, with an oxygen plasma to obtain a surface with aldehyde and carboxyl functionalities. Such a surface can be further modified, for example, by converting the carboxylic acid groups into N-hydroxysuccinimide ester or pentafluorophenyl esters, suitable for further functionalization of the surface with, for example, antibodies. Alternatively, amine based plasma can be used to get an amine terminated diamond like carbon film. These amine terminated surfaces can be reacted with for example alkyl halides, aldehydes, anhydrides and acid halide groups.

A polymeric coating with a covalent coupling to the surface can be obtained through a monolayer with a polymerization initiator, e.g. vinylbenzylchloride or α-bromoisobutyrate, or providing polymerizable groups on the surface for grafting of polymers, e.g., vinyl, acrylate or maleic acid groups. These modified surfaces can be used to create a polymeric layer by polymerization of a monomer based on, e.g., acrylate, acrylamide, methacrylate, methacrylamide, styrene, vinylpyridine, vinyl imidazole or other vinyl monomers, with a hydrophilic, such as a zwitterionic or PEO group, to create a hydrophilic polymeric layer. The polymerization can be done by, for example, free radical polymerization of the monomers and/or controlled living polymerization techniques, such as atom transfer radical polymerization (ATRP) or initiated chemical vapor deposition. Adding crosslinking monomers, e.g., divinylbenzene or ethylene glycol methacrylate during the polymerization may also be beneficial to obtain cross-linked hydrogel layers with increased chemical and mechanical stability.

Zwitterionic polymers can be created by first polymerizing a monomer with a zwitterion precursor functional group, for example, a tertiary amine, pyridine, imidazole group, which can be converted subsequently into a zwitterionic group by chemical reaction. These precursor polymers, for example, poly(dimethyl amino methacrylate), poly(vinyl pyridine) or poly(vinyl imidazole) can be obtained via deposition of the polymer via polymerization from solution, for example, ATRP, or a gas-phase polymerization process, for example, (pulsed) plasma polymerization or initiated chemical vapor deposition directly on the native oxide covered silicon nitride surface or on a native silicon nitride layer obtained by removal of the oxide by hydrogen fluoride etching. Alternatively, the polymer may be grafted on a monolayer providing polymerizable groups on the surface, for example, vinyl, acrylate or maleic acid groups. Subsequently, these polymers may be converted into a zwitterionic polymers by chemical reactions of the tertiary nitrogen atoms in the polymer with, for example, propiolactone, chloro acetic acid, bromo acetic acid, 1,3-propane sulfone, hydrogen peroxide and/or 3-chloroperbenzoic acid.

One of skill in the art will appreciate that such endurable bio or blood compatible coatings in combination with membranes as noted in examples above, according to some embodiments of the present disclosure, are also equally applicable applications other than the capture of CTCs/contaminants from blood, including, for example, blood plasma extraction, leukapheresis, enumeration techniques of water, food, beverage and health borne microbiological contaminants, such as legionella, salmonella, E-Coli, listeria, as well as blood borne bacterial and viral infections. In some embodiments, zwitterionic coatings on perforated silicon nitride or diamond like carbon membranes can also be applied for applications such as for use as a hydrophilic anti-fouling coating on nozzle plates for emulsification, inhalation, spotting, inkjet and other spray applications.

Advantageously, for the selective capture of contaminants from the sample fluid, in some embodiments, the membrane surface (or coating surface if a coating is provided on the surface of the membrane) is provided with antibodies, or more generally stated—affinity bodies or receptor molecules, in combination with a bio-compatible coating. The coating reduces non-specific binding of non-target materials and/or enhances the selectivity of the detection.

For example, a portion or a substantial area of the membrane surface (with or without a coating) can be covered with antibodies, (e.g. CD326) that are able to adhere to at least a part of the CTCs. In this case, the purpose is to create a covalent link of the CTCs to the surface via attachment to small primers attached to the surface with functional group(s), e.g. aldehyde, amine, ester, amide, N-hydroxy succinimide or epoxide. This can also be done in combination with a hemo-compatible coating as described above, for example.

In some embodiments of the disclosure, a membrane (which may also be hereinafter referred to as a filter, a CTC filter, a separation membrane and/or a separation device) is provided which can receive a flow of fluid having CTCs (or other contaminants for capture). Such a bodily fluid may include a viscosity of about 5 milliPa·sec (e.g., blood), which membrane can filter at about 1 ml/min per cm² membrane area at 100 Pa pressure. Thus, about 1 cm² of membrane area is capable of filtering at least 3 ml/min at a pressure of 100 Pa (ca. 1 mbar) for a fluid with a viscosity 5 times higher than water. In some embodiments, a membrane is provided which is capable of removing CTCs (or other similar contaminants) from blood at a flow rate of up to about 10 ml/min/cm², or greater. In some embodiments, the flow capacity of the membrane may be greater than about 40 ml/hour, via a membrane area of about 9 mm2 at a pressure of about 4 torr, yielding a flow rate of about 1 ml/min per cm2, for a bodily fluid having a viscosity of about 5 milliPa-sec. 5 ml/hour at 12 torr 9 mm2. Accordingly, such embodiments enable miniaturized separation devices having high throughput for at least one of in vivo and in vitro applications.

In some embodiments, openings in the membrane can be circular, in the form of slits, as well as other shapes which are advantageous for either or both of the capture of CTCs (and/or other detrimental elements/cells) and passage of necessary and/or healthy components. In some embodiments, slits enjoy the advantage of a greater flux (i.e., The quantity of a fluid that crosses a unit area of a given surface in a unit of time) than other shaped openings. In some embodiments, improved separation of CTCs may occur when the openings of the membrane include a diameter (i.e., width) less than about 8 micrometers, and in some embodiments, even more improved separation results if the openings are less than about 5 micrometers. In some embodiments, slits comprise a shape generally having a length and a width, where the length is longer than the width, and may include an aspect ratio of, for example, of about 10:1 (length to width), with such embodiments rounded or sharply defined corners may be realized. In some embodiments, such slits may comprise a generally rectangular shape, were the corners of such rectangular shape may include a radius. Still other embodiments of the disclosure, include slits which may be elliptical.

In some embodiments, upon the porosity of the membrane (the ratio of the combined surface area of the openings to the total surface area of the membrane including the openings) being at least about 25%, a sufficient minimization of hemolysis, white blood cell (i.e., leukocyte) activation and/or retention and blood platelet activation may be achieved. Also, in some embodiments, high operational flux can be obtained when the nearest centre to centre distance between two openings on the membrane is less than about twice the diameter (width) of the openings—as such this enables the use of a high flux membrane for a miniaturized separation device.

The membrane, according to some embodiments, can retain more than about 85% of CTCs, even when filtering untreated blood (e.g., undiluted, unprocessed, unfixated, etc.). As one of skill in the art will appreciate, in some embodiments, an unexpected advantage has been observed when the thickness of the membrane is between about 1% and about 30%, and preferably, between about 5% and about 25% of the width of the openings in the membrane; for example, in some embodiments, between less than about 0.5 μm and about 2.5 μm (e.g., for openings between about 5 μm and 10 μm); and in some embodiments, between about 0.1 μm and about 0.5 μm. As such, passage of both red and white blood cells in such embodiments is faster when the membrane includes such a thickness. Such a thickness also has been shown to aid in the minimization of at least one of hemolysis, white blood cell activation and/or retention and platelet activation. Such advantages are understood to emanate from increased cell transit times through the openings of the membrane in the above-noted thickness range because of, for example, minimal negative effects on cells passing though the openings.

As such, in some embodiments, the passage of white blood cells may not only be dependent on pore or opening size, shape and/or quantity (e.g., quantity per unit area of membrane), but also on the thickness of the membrane. As noted above, shorter passage times through the openings (and thus, through the membrane), at relatively low trans-membrane pressure, of white blood cells has been observed when the thickness of the membrane is between about, for example, 5% and about 25% of the width of the openings. In such embodiments, substantially all white blood cells (and in some embodiments, all white blood cells) have been able to pass through the openings in the membrane even at a trans-membrane pressure as low as about 1-10 mbar for pores or slits (i.e., openings) with a width of about 3-8 micrometers, while substantial retention (and according to some embodiments, full retention) has been found for CTCs (e.g., epithelial cancer cells). Moreover, it has been found that the vitality of the passed white blood cells is preserved.

In some embodiments, membranes having controlled internal stress are provided. Such membranes are manufactured via a thin film deposition method that leads to an internal stress (of the membrane) at room temperature that is less than about 10% of the maximum yield stress of the material (for example).

It is a particular feature of methods, systems and devices according to some embodiments of the present disclosure, that upon flowing a fluid (bodily fluid or otherwise) containing CTCs for separation by the membrane, that the CTCs are not trapped within the openings/pores of the membrane, but rather, end up along the surface of the membrane (or on the coating if a coating of the membrane is present). Such a feature enables the easy removal of CTCs from a fluid. In some embodiments, this effect is understood to be the result of low viscous (i.e., slippery) leukocytes being available which prevents the CTCs from coming close to the opening entrances—such has been observed at least in experiments where the openings are less than about 5 micrometers, and more specifically, between about 3 and about 5 micrometers. Such permeation and retention results typically are not obtained through the use of relatively thick membranes made of known polymers such as, for example, polyester, polycarbonate, polyimide, nylon and parylene. For thicker membranes, substantial plugging of the openings, especially by white blood cells, has been observed. Accordingly, in some embodiments, such polymeric materials are characterized by relatively small values of Young's Modulus, i.e., a Young's Modulus less than 10 GPa and/or a yield strength less than 1 GPa, and are generally not suited for fabricating mechanically stable and thin membranes according to some embodiments of the present disclosure. Therefore, in some embodiments, the membrane is fabricated from a material with a Young's Modulus greater than about 10 GPa and a yield strength greater than about 1 GPa. In this way, a mechanically stable and relatively thin membrane with high pressure strength can be made—even a membrane having a thickness of only a few hundred nanometers, and more specifically between about 50 and about 500 nanometers. According to some embodiments, a system and/or device for removing CTCs includes at least one membrane (which may be included in a filter assembly and/or housing), an inlet for receiving a bodily fluid from a patient, and an outlet for enabling transfer of such “filtered” bodily fluid back to the patient.

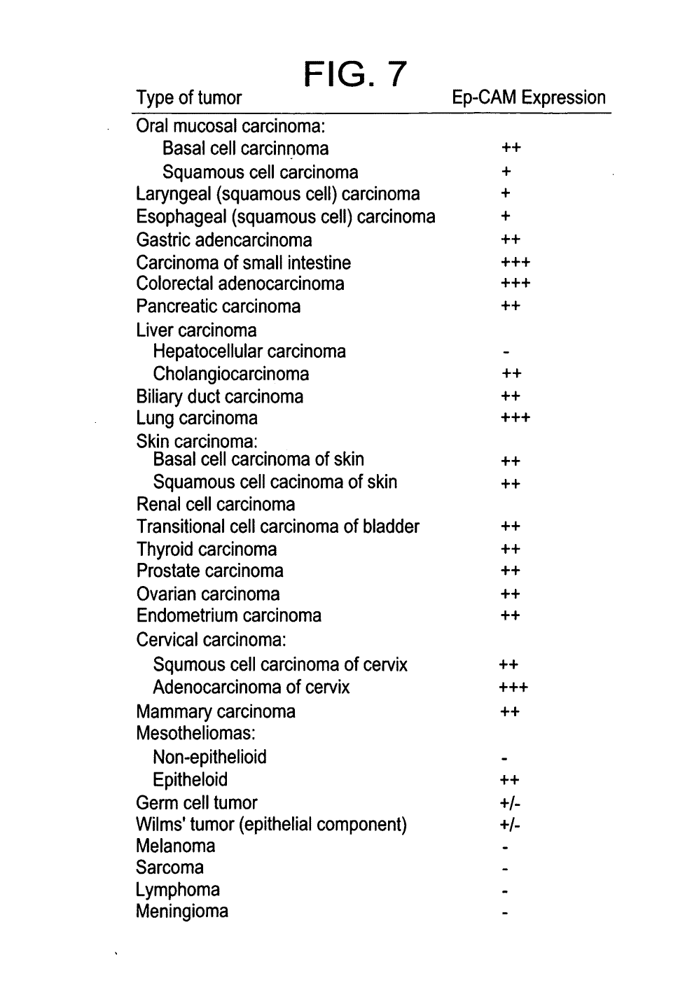

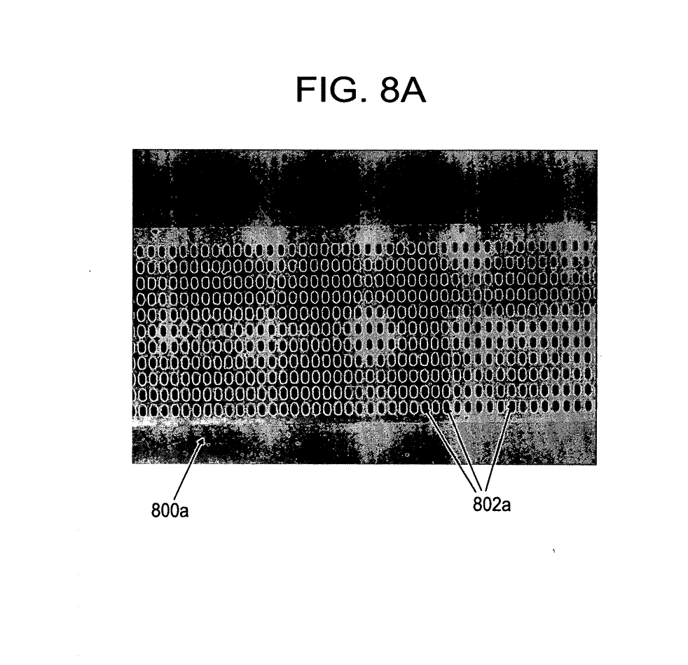

As an example of CTCs, reference is made to Ep-CAM. FIGS. 6A, 6B and 7 illustrate Ep-CAM distribution both in normal (FIGS. 6A and 6B) and cancerous tissues (FIG. 7). Referring to FIGS. 6A and 6B, the level of Ep-CAM expression by specific cell types within epithelial tissues is indicated by “−” for no expression, “+” for low expression levels, “++” for intermediate expression levels and “+++” for high expression levels. Referring to FIG. 7, most carcinomas express Ep-CAM, whereas tumors derived from non-epithelial tissues are Ep-CAM negative. The level of Ep-CAM expression by tumor cells is indicated by “−” for no expression, “+” for low expression levels, “++” for intermediate expression levels and “+++” for high expression levels. Ep-CAM is a strictly epithelial molecule in adult humans and detected at the basolateral cell membrane of all simple, pseudo-stratified, and transitional epithelia. See Balzar, M., et al., “The Biology of the 17-1A antigen (Ep-CAM),” J. Mol. Med., 77:699-712 (1999). This reference is herein incorporated by reference in its entirety. Many carcinomas express high levels of Ep-CAM (see FIG. 7). Balzar at 704.

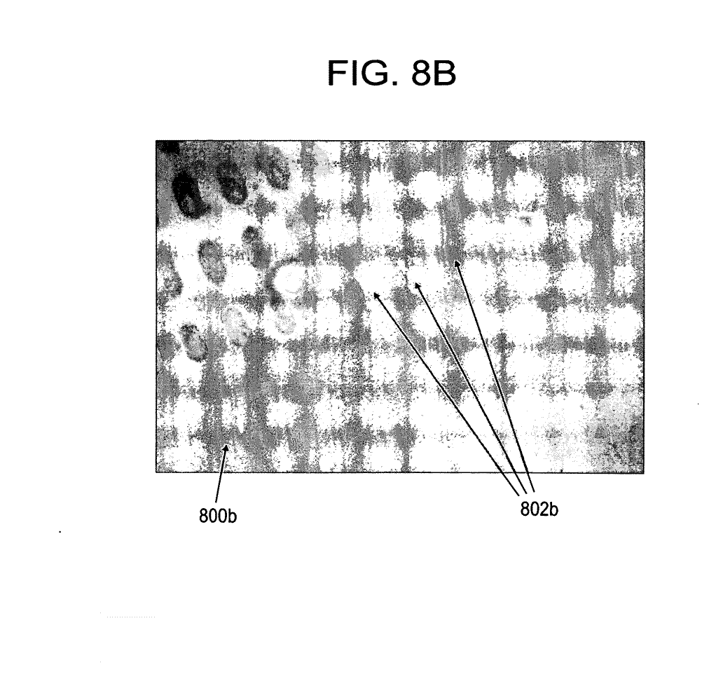

FIGS. 8A and 8B are enlarged photographs of membranes according to some embodiments of the disclosure. Accordingly, membrane 800a, 800b is shown (at different magnifications, FIG. 8B being at greater magnification), having orderly (for example) slits shaped openings 802a, 802b (for example), where the corners may include a radius.

### Example 1

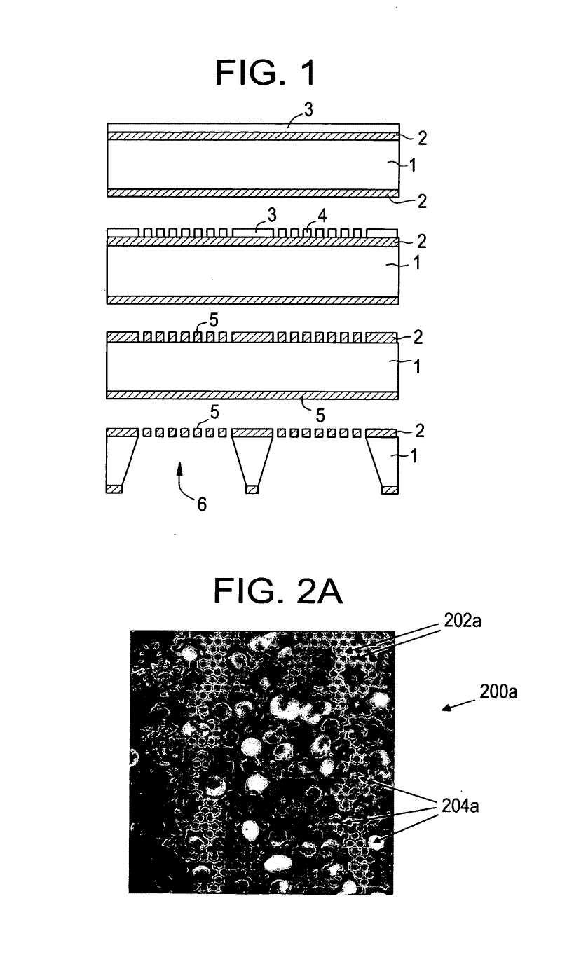

With reference to FIG. 1, using a monocrystalline silicon wafer 1, a silicon rich silicon nitride membrane is made with openings with a pore size of 5 micrometer (see FIG. 1). The silicon nitride membrane comprises a layer 2 having a thickness of 400 nanometers, and is deposited on a 750 μm thick polished silicon wafer 1 by means of, for example, a low pressure chemical vapor deposition process (LPCVD) leading to a relatively low internal tensile stress (e.g., by choosing the ratio of silicon and nitride in a controlled manner during the deposition). In some embodiments, the obtained silicon rich silicon nitride layer includes an elastic modulus of about 290 GPa and a Yield stress of about 4 GPa. Next, a photo-resistive layer 3 is formed by spin-coating. This layer is patterned with pores 4 having a diameter of about 5 micrometers, and is produced by exposing the membrane to UV light through a photo mask (for example). The pattern in the photosensitive layer 3, 4 is transferred on/in the silicon nitride membrane 5 by means of, for example, Reactive Ion Etching (RIE) and thus, openings 5 are formed in the membrane. Finally, the monocrystalline 100 silicon body is anisotropically etched with large through holes 6 with deep reactive ion etching (according to some embodiments). Alternatively, a boron doped diamond like carbon membrane (DLC) can be obtained using a hot filament chemical vapor deposition method and a boron doped ethyl alcohol precursor. Etching of the pores in the DLC film may be carried out using a silicon dioxide mask. Typical values for the obtained DLC film includes an elastic modulus of greater than about 100 GPa and a Yield stress of greater than about 2 GPa.

The processed silicon wafer is then provided with a zwitterionic coating of approximately 30 nanometers thick, for example, with sulfobetaine groups obtained with known chemical methods, and is covalently attached to the silicon nitride. The processed wafer is then treated with an oxygen plasma and subsequently reacted for about 2 hours with an alkoxy silane solution of about 2.5% (3-trimethoxysilyl)propyl 2-bromo-2-methylpropionate in ethanol. The wafers are then taken out of the solution and rinsed with ethanol and dried under an argon flow. The polymer is then grafted from the surface using, for example, atom transfer radical polymerization. A solution of sulfobetaine methacrylamide monomer and bipyridine ligand in isopropanol/water (3/1) is purged with argon for 20 minutes and added to CuBr under argon atmosphere. The CuBr solution with monomer and ligand is added to the initiator coated wafer (under argon atmosphere) and the polymerization reaction is allowed to proceed for three hours. The wafer is taken out of the solution and rinsed with clean warm water/isopropanol mixture and dried under an argon flow. Alternatively, the processed silicon wafer is provided with a titanium dioxide coating having a thickness of approximately 10 to 50 nanometers. The completed wafer is cut in chips, each having a size of about 10×25 mm (for example). Each chip contains about 1.25 million pores with a diameter of about 5 micrometers.

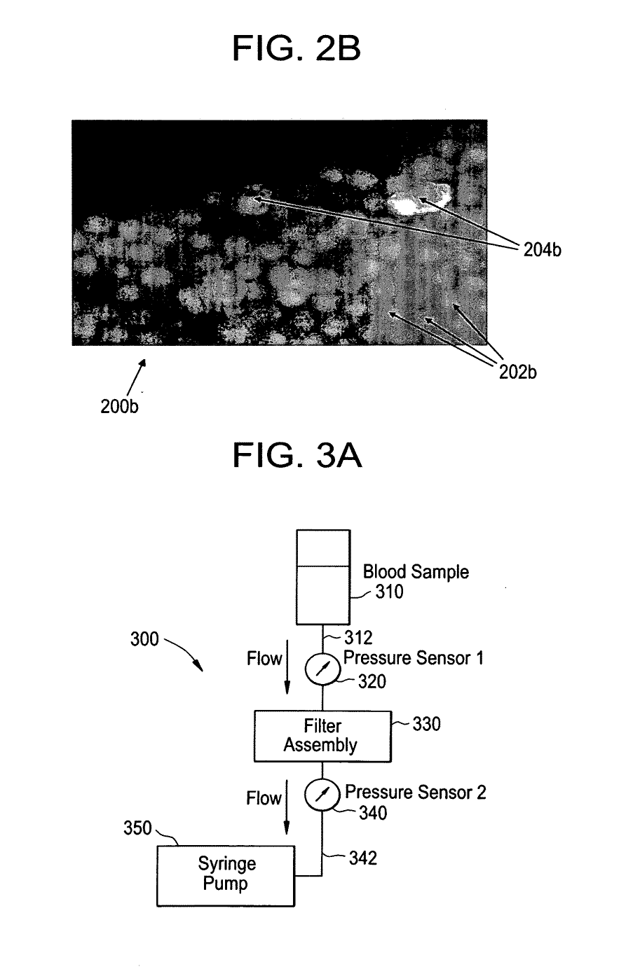

Accordingly, in a method for removing CTCs from a fluid, 1,500 prostate epithelial cancer cells (tumor cells) were purposely added to 500 ml of blood from a healthy volunteer and pressed at low pressure through one of the above-noted filters (e.g., an assembly or module including a membrane, e.g., 200a, 200b, according to some embodiments disclosed herein; such membranes may be packages and/or referred to as a filter, membrane, and/or membrane chip) in a dead-end mode for about 15 minutes with use of a filtration module. The measured hemolysis of blood passed was less than 0.1%, passage of white blood cells was greater than 99.9% and recovery of blood platelets was greater than 99.999%. The membrane chip was then removed from the module and the cells collected at/on the membrane were re-suspended in 400 μl of a buffer containing the UV excitable nucleic acid dye DAPI (Molecular Probes) and Cytokeratin monoclonal antibodies (identifying epithelial cells) labeled with the fluorochrome Cy3. After a washing step, the membrane chip was viewed at 20× magnification for the presence of the tumor cells 204a, 204b (see, e.g., FIG. 2A). At least 1,450+/−50 cells were identified using fluorescence spectroscopy. It was observed that the silicon nitride membrane was free of auto-fluorescence and that the membrane was flat and easily brought into the focus plane of the microscope. In the embodiment utilized in this Example 1, the absence of leukocytes in the membrane openings 202a, 202b (see, e.g., FIG. 2A) may be attributed to the use of the zwitterionic coating (e.g., without this coating, leukocytes 202b may be present in many membrane openings.

Accordingly, as exemplified by the above example, some embodiments of the present disclosure also provide methods, systems and devices to separate and count CTCs, and in particular, can also be used for diagnosis or during therapeutic treatment, using, for example, thin and mechanically flat and stable membranes. Cell counting can be further optimized by using membranes that have been functionalized with antibodies (e.g. CD326) that are able to adhere to the CTCs.

### Example 2

CTC Enumeration. Approximately 10 prostate epithelial cells were purposely added to 8 ml of blood from a healthy volunteer and flowed at a low pressure of 4 torr through a 3 mm×3 mm membrane chip with 20,000 slit shaped pores (5×10 micrometers) in a dead-end mode for approximately 15 minutes with use of a filtration module. After filtering, the membrane chip was washed with 10 ml PBS in dead end mode. Next, a 2% formaldehyde in PBS for 5 minutes was used to fixate captured cells. The following washes took place.
wash with 10 ml PBS;
wash 1 ml 0.2% Triton X-100 in PBS to induce cellular permeability;
wash with BSA blocker to prevent non-specific adsorption of antibodies);
wash 1 ml anti-CD45 solution (50 μl of CD45-APC stock in 1 ml PBS)
10 ml PBS wash step.
1 ml anti cytokeratin (50 μl anti-CK-PE stock in 1 ml PBS)
10 ml PBS wash;
wash 1 ml DAPI solution; and
10 ml PBS wash.

The membrane was then stored at about 4° C. until imaging. Using fluorescence microscopy, it was found that all prostate cancer cells that were added to the blood sample were retrieved.

### Example 3

CTC Enrichment for gene therapy. Blood (8 ml) from a patient is fed through a membrane chip with 40,000 slit shaped pores (having dimensions of about 3×10 micrometers) in a dead-end mode for about 15 minutes with use of a filtration module to collect about 10 CTCs. In order to perform DNA analysis on the collected CTCs without disturbance of other DNA of healthy blood cells, the cells on and in the membrane filter was controlled by one or more of the following steps:
the membrane filter was washed with 10 ml PBS in dead end mode;
captured cells were put in a hypotonic solution to allow swelling of the cells. Cells (typically white blood cells) that are inside the pores get trapped, whereas CTCs on top of the membrane can be rinsed off quite easily for further DNA analysis;
the membrane filter is provided with an anti sticking coating (PTFE, TiO2, Zwitterionic, PEO, HEMA) in order to push out all white blood cells located in the pores using a hypertonic solution that shrinks cells; and
white blood cells are fixated with selective labeling with AB labeled with magnetic beads.
the membrane filter is provided with magnetic beads having EPCAM (CD326) that couples to the CTCs. Next with the use of a magnet the CTCs are taken away from the membrane surface for further interrogation

### Example 4

CTC Clearance of patient's blood. Blood from a patient is led through a membrane chip, or an array of membrane chips, with a cumulative surface area of about 10 cm², with slit shaped pores (having, for example in this case, a slit shaped pore size of 3×10 micrometers) in a dead-end mode for about 60 minutes with use of an extracorporeal filtration module to collect virtually all of the patient's CTCs. At 12 torr trans-membrane pressure, the mean flow rate of patients blood is 2.0 liter/hour/10 cm². Accordingly, a long session (e.g., 1-2 hours) capable of clearing a patient's entire blood volume of CTCs can be either performed in a clinic or ambulatory setting. An anti-coagulant, as known in the art, (conf. plasma pheresis) can be added during the session depending on the specific requirements, though as indicated earlier in the disclosure, it is not required in some embodiments. After the long session, a significant quantity of CTCs can be obtained in this way for gene therapy and other treatment modalities.

In general, cell separation utilizing a membrane according to some embodiments of the disclosure, may be determined by at least one of (and in some embodiments, several of, and in some embodiments, all of) diameter/size of the openings, thickness of the membrane, and density of openings/pores arranged on the membrane (among other characteristics), and by the biochemical interactions between the cell and material surfaces, including, for example, cell adhesive capacity on the membrane surface, and/or such capacity using a coating.

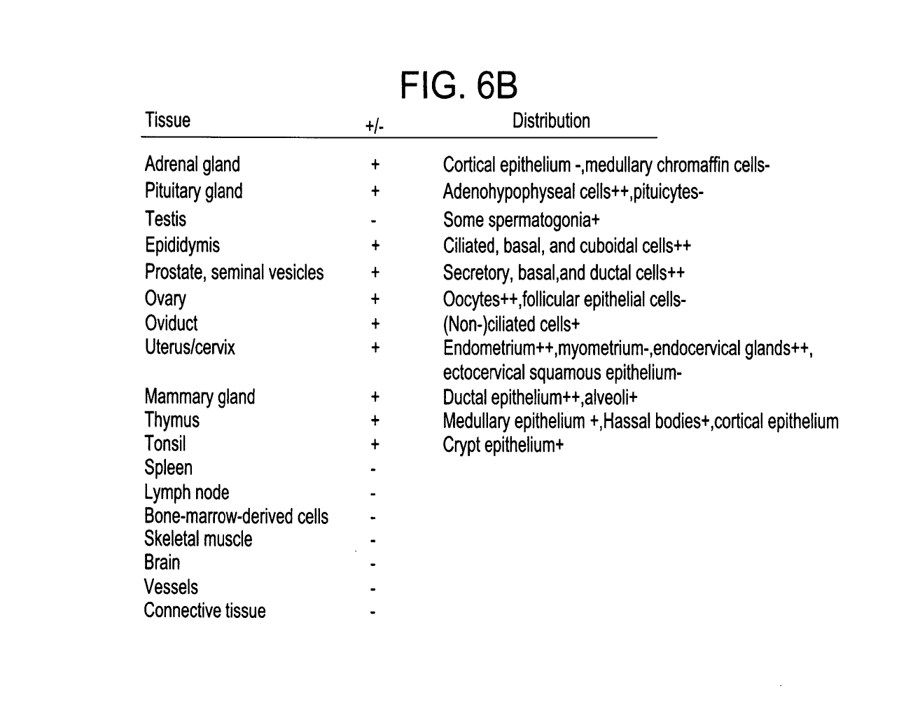

In some embodiments of the present disclosure, an automated system using one or another of the CTC collection chips discussed above for capturing and/or collecting CTCs is provided. Such systems include a control process (which may also be referred to as a control algorithm) having predetermined inputs, outputs, and control parameters. FIG. 5 illustrates a high-level diagram of the control process according to some embodiments of the present disclosure.

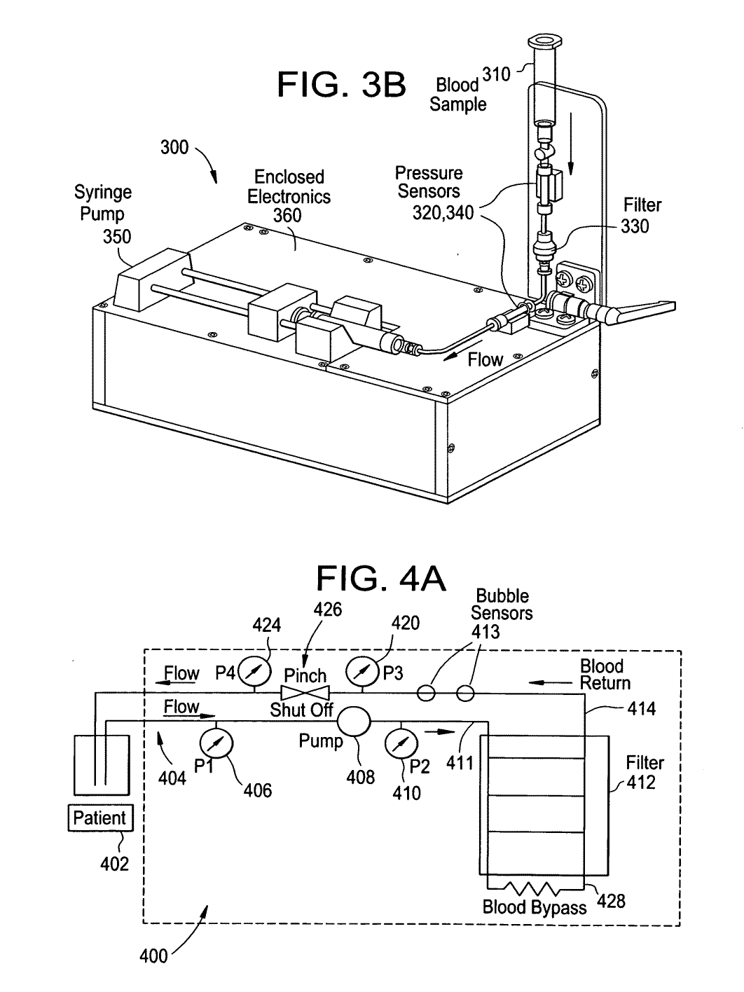

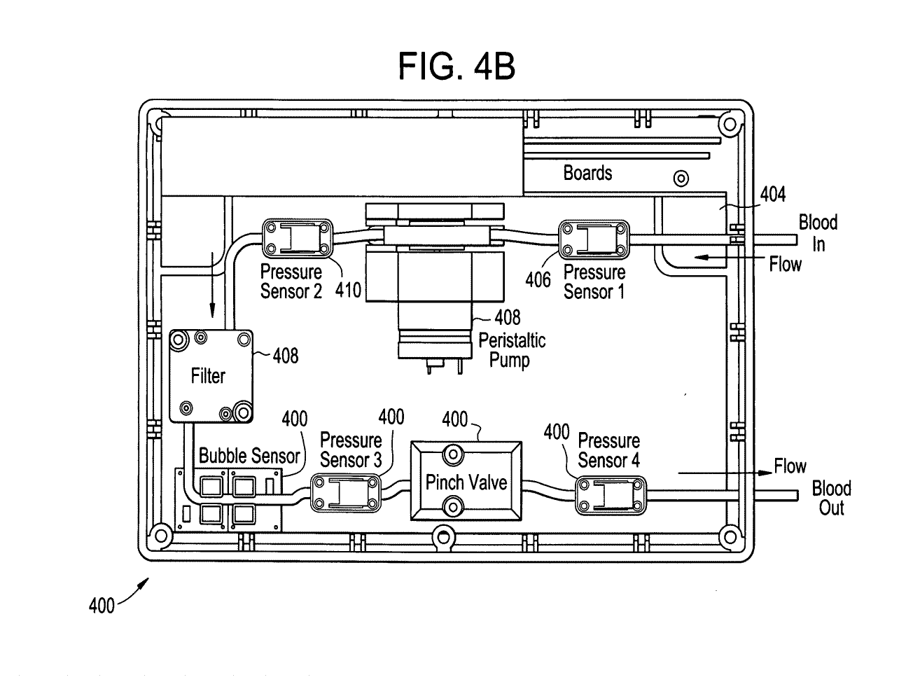

FIGS. 3A-B and 4A-B are schematic diagrams and perspective mockups of exemplary systems, according to some embodiments of the present disclosure, for capturing CTCs which include CTC filters according to one or another of such CTC filter embodiments of the present disclosure. FIGS. 3A-B illustrate a system 300 which includes a limited fluid sample 310 (e.g., blood), which can be provided within a sample container (e.g., syringe body, as shown in FIG. 3B), in fluid communication via a fluid conduit 312 with a filter assembly 330, which includes a CTC filter (e.g., see FIG. 1) for separating CTCs from the sample, first pressure sensor 320, for monitoring an input pressure to the CTC filter, is located before the CTC filter 330 (comprising, for example, a membrane according to some embodiments) in the direction of fluid flow provided along a portion of the fluid conduit 312, second fluid sensor 340, for monitoring output pressure from the CTC filter, is located after the CTC filter in the direction of fluid flow along a portion of the fluid conduit 342, and a syringe pump 350. The syringe pump applies negative pressure to an end of the fluid conduit 342 so as to draw the fluid sample containing the CTCs out of the sample container, through the CTC filter, and various fluid conduits and pressure sensors, where it is then collected as a filtered fluid within a container (e.g., syringe body) of the syringe pump. Various electronics 360 (not shown in FIG. 3A) may also be provided, including (but not limited to) controllers, processors, regulators, circuitry, sensors, communications (e.g., wifi, Bluetooth, cellular and/or wire connection—e.g., Ethernet), memory, and power supply (e.g., batteries, AC and/or DC power supply), hereinafter referred to as “Various Electronics” (which may be one or more of the described components, but is not limited to such described components); such may be arranged as depicted in FIG. 3B, for example, in a compartment. The Various Electronics may be provided to at least one of monitor, control, communicate (to and/or from the system) and supply power to the system. One of skill in the art will appreciate that other types of pumps may be used to draw and filter fluid samples, including but not limited to peristaltic pumps, gear pump, progressive cavity pumps, roots-type, venturi pump, piston/reciprocating pumps, compressed gas/air pumps, and the like.

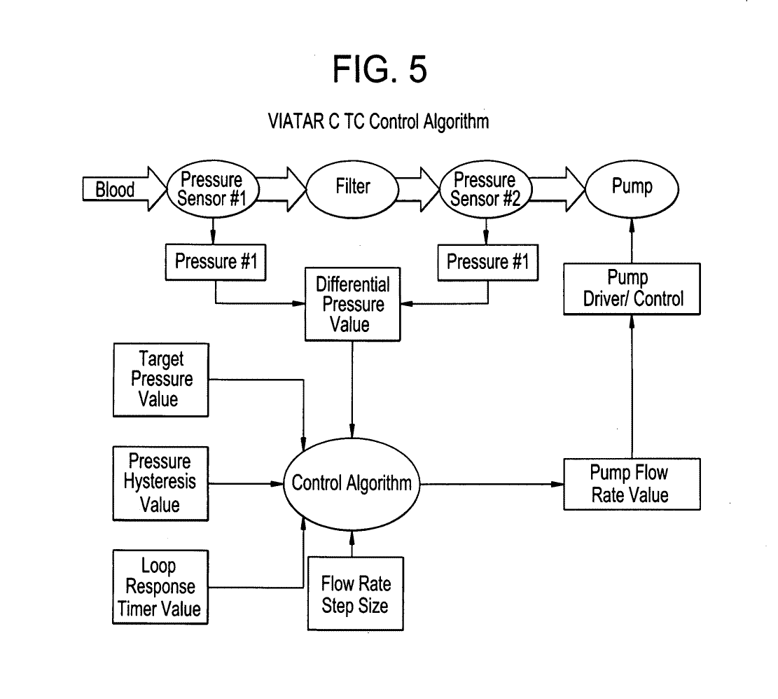

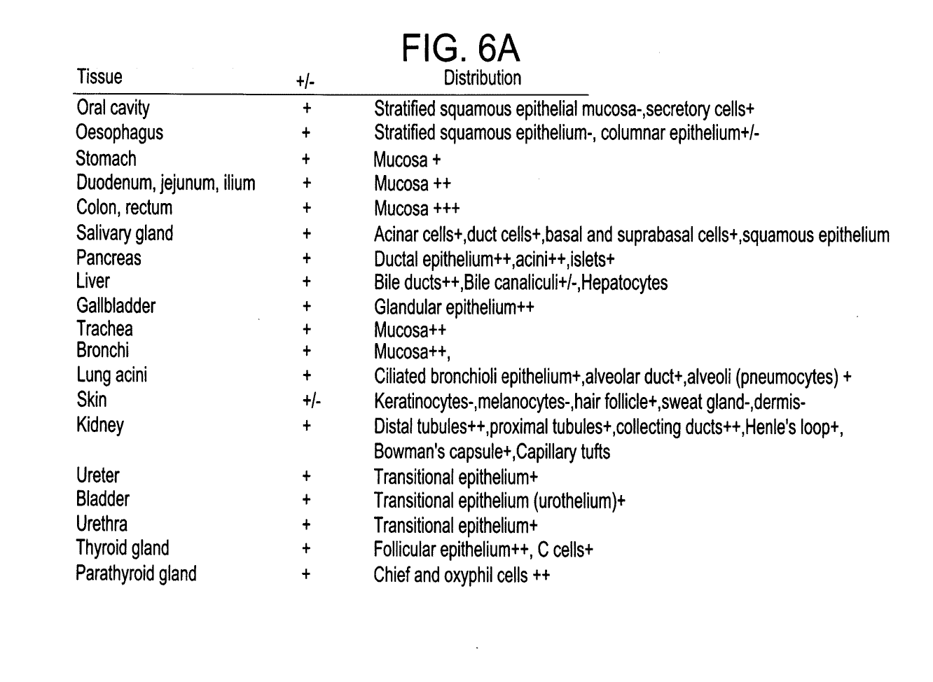

FIGS. 4A-B illustrate an extracorporeal system 400 according to some embodiments, for removing CTCs from the blood of a patient, directly from the patient. In some embodiments, such a system can be used to remove CTCs from substantially all of a patient's blood (and, preferably, all of a patient's blood). Accordingly, bodily fluid 402 (e.g., blood) from a patient is directed along a fluid communication path 404 to pump 408 (e.g., peristaltic pumps, gear pump, progressive cavity pumps, roots-type, venturi pump, piston/reciprocating pumps, compressed gas/air pumps and the like). Pressure sensor 406 for monitoring a pressure P1 (input pressure of the CTC filter) is provided along the fluid conduit before the pump in the direction of flow, and pressure sensor 410 for monitoring pressure P2 (output pressure of the CTC filter) is provided along fluid conduit 411 after the pump in the direction of flow. The bodily fluid 402 is then directed to the CTC filter 412 where the CTCs are removed from the flow. Thereafter, the blood is returned to the patient via conduit 414. At least one bubble sensor 413 may be provided along a portion of the conduit (e.g., along conduit 414), and in some embodiments, two such bubble sensors can be provided. Pressure sensor 420 which provides an indication of pressure P3, and pressure sensor 424 which provides an indication of pressure P4. In some embodiments, between pressure sensor 420 and 424, a valve 426 (e.g., a pinch shutoff valve) may be provided. From that point, the filtered bodily fluid is then directed back to the patient for incorporation into the patient (e.g., into the patient's bloodstream). A bypass 428 may also be provided to direct all or a portion of the flow around the filter. As described in the embodiments shown in FIGS. 3A-B, Various Electronics may be provided to at least one of monitor, control, communicate (to and/or from the system) and supply power to the system.

According to such embodiments, for example, that which is depicted, for example, in FIGS. 3A-B:
input pressure may be measured in mmHg at the input side of the CTC filter (e.g., filter-chip);
output pressure may be measured in mmHg at the output side of the CTC filter/chip (which may be prior to the pump, for example, in a limited sample system; e.g., see FIGS. 3A-B);
differential pressure value is the calculated difference in pressure measured by input and output pressure sensors to the filter (e.g., 320, 324)
target pressure value is the differential pressure to be achieved and preferably maintained across the CTC filter/chip during a separation process;
pressure hysteresis value is the absolute pressure deviation allowed across a CTC filter/chip prior to pump flow rate adjustment;
loop response timer value represents a minimum time between consecutive automated control process executions; in essence, the timer is started upon completion of the initial automated control process and terminates prior to any further executions of the process;
flow rate step size is the value by which the pump flow rate value is modified between consecutive executions of the automated control process executions; and
pump flow rate value is the automated control process output used to set the system pump flow rate.

Accordingly, in some embodiments, an important function of an automated control process for separating (i.e., filtering) out CTCs is to maintain a constant differential pressure across the CTC filter. To that end, a CTC control process according to some embodiments enables such functionality by performing at least a plurality of the following steps, and in some embodiments, all the following steps. An embodiment of the control process is illustrated in FIG. 5.

A bodily fluid containing CTCs is processed by the system. Filter input and filter output pressures (e.g., via input pressure sensor 320, and via output pressure sensor 340) are measured, in mmHg, at predetermined time intervals. Utilizing these values, the differential pressure value is calculated. The input differential pressure value is then compared to the calculated (predetermined) target pressure range. The target pressure range may be comprised of the target pressure value±pressure hysteresis value. If the input differential pressure value is within the target pressure range, the CTC control process terminates without modification to the pump flow rate value and jumps back to step 1; otherwise a new pump flow rate value will be calculated as defined below.

As stated previously, one of the goals of the CTC control process is determine and, if necessary, update a new pump flow rate value such that the system maintains a substantially constant pressure value across the CTC filter without overshoot—i.e., not exceeding a target differential pressure by more than a specified percentage (or hysteresis). To accomplish this, the pump flow rate value is preferably updated on a periodic basis to minimize pressure overshoot. To accomplish this goal, the CTC control process execution may be limited according to the value configured in a pump loop response timer. The timer, which counts down to zero, is intended (according to some embodiments) to limit and/or block execution of the control process. Once the timer terminates, the control process launches and initially determines at least one of (and preferably both of) the range of pressure error and its corresponding direction (e.g., positive or negative). Once the pressure error and/or direction are calculated, the CTC control process then determines a new pump flow rate value according to one of the following mathematic formulas:

differential pressure value>(target pressure value+pressure hysteresis value). Equation (1):

In the case of equation (1), the present pump flow rate value will be reduced by the flow rate step size.

differential pressure value<(target pressure value−pressure hysteresis value). Equation (2):

In the case of equation (2), the present pump flow rate value will be increased by the flow rate step size.

In some embodiments, the flow rate step size is selected to eliminate overshoot, though this may result in pressure oscillation. Upon completion, a controller of the pump is updated with the updated pump flow rate value which updates the actual pump speed. The process is then repeated.

In some embodiments, the noted CTC control process substantially avoids (and preferably, fully avoids) hemolysis of blood and plugging of the openings/pores of the membrane by leukocytes. It is also worth noting that in some embodiments, methods, systems and devices for filtering CTCs from blood can operate either with or without anticoagulants (e.g., heparin, citrate).

Some embodiments of the present disclosure provide a membrane having a predetermined number of pores for a given quantity of blood. In such embodiments, it can then be ensured that should CTCs end up by chance over an opening, only an insignificant number of openings are blocked (with CTC). For example, capturing 10,000 CTCs may required greater than 1 million pores.

Any percentages listed according to various embodiments (e.g., majority percentage of captured CTCs, passed leukocytes, etc.), also include percentages in between those listed.

While some embodiments have been described as single-flow systems, parallel and serial arrangements of various embodiments presented are also within the scope of the present disclosure. Accordingly, processing times for capture of CTCs (for example) from a bodily fluid can be shortened utilizing a parallel flow arrangement. Moreover, a serial arrangement may also be provided for capture at least one type of CTC (and/or contaminant, cell, etc.), and subsequent filters set up to capture the at least one type of CTC, and/or other types of CTCs and/or contaminants. Thus, such features can be a part of any of the disclosed embodiments.

Implementations of various embodiments disclosed herein (e.g., extracorporeal systems) may be realized utilizing controllers and other electronic means/processors including, for example, digital electronic circuitry, integrated circuitry, specially designed ASICs (application specific integrated circuits), computer hardware, firmware, software, and/or combinations thereof. Such embodiments may include implementations in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, for example, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user (e.g., patient, healthcare worker), some embodiments may include implementation via a computer having a display device (e.g., a CRT (cathode ray tube) or LCD (liquid crystal display) monitor and the like) for displaying information to the user and a keyboard and/or a pointing device (e.g., a mouse or a trackball) by which the user may provide input to the computer. For example, such a program can be stored, executed and operated by the dispensing unit, remote control, PC, laptop, smart-phone, media player or personal data assistant (“PDA”). Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

Some embodiments of the present disclosure may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

Accordingly, a computing system according to some such embodiments described above may include clients and servers. A client and server are generally remote from each other and typically interact through a communication network. The relationship of client and server arises by virtue of computer programs running on the respective computers and having a client-server relationship to each other. For example, a patient that does not have a controller “at arm's length”, can administer and control certain functionality of various method, system and device embodiments described herein via the internet. Other embodiments include methods, systems and devices which include a physician or healthcare worker that is located far from the patient (and system/device), but still able to monitor, operate and receive data from the device via the internet or a data server, e.g., a U.S. based physician can communicate with the device and patient which are situated overseas.

Any and all references to publications or other documents, including but not limited to, patents, patent applications, articles, webpages, books, etc., presented in the present application, are herein incorporated by reference in their entirety.

Although a number of embodiments have been described in detail above, other embodiments and modifications to disclosed embodiments are possible. For example, the logic flow depicted in accompanying figures and described herein does not require the particular order shown, or sequential order, to achieve desirable results.

It is worth noting that any and all features and any and all functionality among various disclosed embodiments may be mixed and matched among various embodiments to present other embodiments, which may be either within the scope of one or another of the appended claims, and/or within the scope of claims subsequently presented in this and/or subsequently filed in related applications. Accordingly, it is understood that the embodiments and examples described herein are for illustrative purposes only, and that various and many modifications will be suggested to persons skilled in the art and are to be included within the scope of the disclosure of this application. While at least some of the disclosed embodiments are included within the scope of the appended claims, Applicants also reserve the right to pursue other claims for the subject disclosure in either or both of the present and subsequent applications claiming benefit of the subject application. Such claims may include claims similar to the appended claims, including broader aspects of such embodiments currently claimed, as well as other any and all aspects, embodiments and inventions disclosed, taught and/or otherwise supported by the present disclosure.

1-45. (canceled)

46. A method of separating circulating tumor cells from blood cells in a bodily fluid using a filter membrane that has a first side, a second side opposite the first side, and a plurality of pores extending from the first side to the second side, the blood cells including leukocytes, the method comprising:
contacting the bodily fluid with the first side of the filter membrane; and
applying a pressure differential across the filter membrane such that at least a majority of leukocytes in the bodily fluid pass through the pores to produce a filtrate at the second side of the filter and such that at least 75% of the circulating tumor cells are retained at the first side of the filter membrane,
wherein the leukocytes that pass through the filter membrane retain their vitality,
each of the pores of the filter membrane has a minimum dimension, in a plane of the first side, of between 3 μm and 8 μm,
the filter membrane has a thickness in a direction from the first side to the second side that is less than the minimum dimension of each pore.

47. The method of claim 46, further comprising returning the filtrate and blood cells therein to a patient.

48. The method of claim 46, wherein the blood cells at the first side and in the filtrate are non-lysed whole cells.

49. The method of claim 46, wherein the pressure differential is less than or equal to 12 Torr.

50. The method of claim 46, wherein the blood cells include red blood cells, and the applying a pressure differential is such that hemolysis of the red blood cells after passing through the filter membrane is less than 1%.

51. The method of claim 46, wherein each of the pores has a circular opening in the plane of the first side, and the minimum dimension comprises a diameter of the circular opening.

52. The method of claim 46, wherein each of the pores comprises a slit in the plane of the first side, with a ratio of length to minimum dimension of each slit being greater than one with a shape that is either rectangular or elliptical and a width between 3 and 8 microns.

53. The method of claim 46, wherein the minimum dimensions of the pores differ by no more than 0.5 μm from each other.

54. The method of claim 46, wherein the filter membrane thickness is between 5% and 25% of the minimum dimension of each pore.

55. The method of claim 46, wherein the filter membrane thickness is less than 2.5 μm.

56. The method of claim 46, wherein the applying a pressure differential is such that at least a predefined percentage of the leukocytes in the bodily fluid at the first side are passed through the filter membrane to the second side where the predefined percentage is greater than about 75%, greater than about 80%, greater than about 85%, greater than about 90%, greater than about 95%, greater than about 99%, or greater than about 99.9%.

57. The method of claim 46, wherein the applying a pressure differential is such that a flow rate through the filter membrane is at least 1 ml/min per cm² of surface area of the first side of the filter membrane.

58. The method of claim 46, wherein the applying a pressure differential is such that a flow rate through the filter membrane is between 1 and 10 ml/min per cm² of surface area of the first side of the filter membrane

59. The method of claim 46, further comprising monitoring a first pressure at the first side and a second pressure at the second side, wherein the applying is responsive to the monitoring so as to maintain a constant pressure differential across the filter membrane.

60. The method of claim 46, wherein the filter membrane comprises a base material having a Young's Modulus greater than 10 GPa and a yield strength greater than 1 GPa.

61. The method of claim 46, wherein the coating is less than 500 nm thick.

62. The method of claim 46, wherein the bio-compatible coating comprises one or more zwitterionic polymeric materials, and the zwitterionic polymeric materials comprise phosphorylcholine, sulfobetaine, carboxybetaine, amine-N-oxide sub groups, or combinations thereof.

63. The method of claim 46, further comprising covalently linking the circulating tumor cells to antibodies attached to a surface.

64. The method of claim 63, wherein the antibodies are covalently linked to a surface of the filter membrane at said first side.

65. The method of claim 46, wherein the filter membrane has a coating of, or is formed of, a biocompatible or blood-compatible material.

66. The method of claim 46, wherein the filter membrane has a coating of a biocompatible or blood-compatible material.

67. The method of claim 46, wherein the filter membrane has a coating of, or is formed of, a biocompatible or blood-compatible material.

68. A method comprising:
contacting with a first side of a filter membrane a fluid containing blood cells and circulating tumor cells from a patient, the blood cells comprising non-lysed whole cells,
the filter membrane having a second side opposite the first side and a plurality of pores extending from the first side to the second side, each of the pores having a minimum dimension, in a plane of the first side, of between 3 μm and 8 μm,
the filter membrane having a thickness in a direction from the first side to the second side that is less than the minimum dimension of each pore,
applying a pressure differential across the filter membrane such that blood cells pass through the pores to produce a filtrate at the second side of the filter and such that at least 75% of the circulating tumor cells are retained at the first side of the filter membrane; and
returning the blood cells without the retained circulating tumor cells to the patient, the blood cells retaining their vitality after the applying a pressure differential.

69. The method of claim 68, wherein the blood cells from the patient include leukocytes and the applying a pressure differential is such that at least a majority of leukocytes at the first side pass through the pores and retain their vitality.

70. The method of claim 68, wherein the filter membrane has a coating of, or is formed of, a biocompatible or blood compatible material.

71. The method of claim 68, wherein the filter membrane has a coating of a biocompatible or blood compatible material.

72. The method of claim 68, wherein the blood cells include red blood cells, and the applying a pressure differential is such that hemolysis of the red blood cells after passing through the filter membrane is less than 1%.

73. The method of claim 68, wherein the filter membrane thickness is less than 2.5 μm.

74. The method of claim 68, wherein the applying a pressure differential is such that a flow rate through the filter membrane is at least 1 ml/min per cm² of surface area of the first side of the filter membrane.

75. The method of claim 68, wherein the bio-compatible coating comprises one or more zwitterionic polymeric materials, and the zwitterionic polymeric materials comprise phosphorylcholine, sulfobetaine, carboxybetaine, amine-N-oxide sub groups, or combinations thereof.

76. The method of claim 68, further comprising covalently linking the circulating tumor cells to antibodies attached to a surface.

77. The method of claim 68, wherein the antibodies are covalently linked to a surface of the filter membrane at said first side.

78. The method of claim 68, wherein the applying a pressure differential is such that a flow rate through the filter membrane is between 1 and 10 ml/min per cm² of surface area of the first side of the filter membrane.
